(19) United States
(12) Patent Application Publication
(10) Pub. No.: US 2004/0245281 A1
(43) Pub. Date: Dec. 9, 2004

(54) Beverage line purifier

(76) Inventor: Simon Fobes Oke, Wingfield, South Australia (AU)

Correspondence Address: FINNEGAN, HENDERSON, FARABOW, GARRETT & DUNNER, LLP, 1300 I STREET, NW, WASHINGTON, DC, 20005, US

(21) Appl. No.: 10/480,533

(22) PCT Filed: Jun. 13, 2002
(86) PCT No.: PCT/AU02/00761

(30) Foreign Application Priority Data
Jun. 13, 2001 (AU) PR 5633

(51) Int. Cl. B67D 1/08

(52) U.S. Cl. 222/1; 222/148

(57) ABSTRACT

A method and apparatus for cleaning and purifying beverage lines such as beer lines. Oxidants and oxidant radicals are produced electrically in a stream of air and the resultant gas is injected into a stream of water to pass slowly through the lines to be cleaned and purified.

### BACKGROUND OF THE INVENTION

It is well known that beverage lines and vessels need to be regularly cleaned. Cleaning is also referred to as purifying or sanitising or disinfecting. Lines are also referred to as pipes or tubes or hoses or conduits or fittings or faucets or dispensing systems. Vessels include tanks, vats, kegs and other containers. Draft beer is the most common liquid for this application because of the tendency for yeast build-up, but other beverages include non-beer liquids such as water, milk, wine, syrup, juices, colas, sodas, carbonated drinks, and post-mix lines where syrup is converted to carbonated drinks. The line is cleaned between its source (for example a keg or storage vessel, and its end point (for example a beer tap or spigot or dispensing machine). The organisations in which this occurs includes hotels, clubs, pubs, breweries, dairies, wineries and other organisations where beverage is produced or transported or consumed.

Hotels for example, often store beer in kegs in a basement floor and then pump it up to dispensing taps at the bar at the floor above. The length of the beer lines can be substantial and the number and complexity of beer lines per organisation can also be large.

When beverage is moved through a line or stored in a line, substances are deposited on the inner surfaces of the line. These may be called pollutants because the objective is to keep the inner surface of the line pure and clean, and may include yeasts, calcium oxalate, beer-stone, milk-stone, bio-film, bacteria, protozoa, trichloroanisole (TCA) and other pollutants. These substances must be removed periodically. Otherwise the line becomes unhygienic and will detrimentally affect the beer being transported through it. Also the pressure drop can increase and affect the draw or flow rate of beverage.

Many organisations therefore clean and purify their beverage lines between once per day and once per month, The word “clean” may be used to refer to the removal of deposits and debris and scale from the inside of the line. The word “purify” may be used rather differently to refer to the killing of microbes on the inside of the line. Therefore it is necessary to achieve both objectives to clean and to purify. Beer lines are typically cleaned weekly, dairy lines are typically cleaned daily, whilst winery lines are also cleaned regularly.

Beer Line Purifier products and processes are well known. Most use liquid chemicals which soak in the line. Others use small projectiles which are moved through the line by using compressed air and thus scour and clean the inside of tile line.

A common process is as follows. The organisation purchases suitable chemicals, which can include caustic based solutions, detergents, defoamers, chelating agents, alkali salts, iodine, acids, etc. These are poured into a vessel (such as a spare empty keg) and then diluted or mixed with water. The inlet of the vessel is connected to the carbon dioxide bottle system (which typically exists in the hotel for the transportation of beer through the lines). The outlet of the vessel is connected to the beer line, which is to be cleaned. A valve is then opened to allow the carbon dioxide to displace the chemical mixture out of the vessel and to fill the beer line to be cleaned. The chemical is left static in the line for a period of time (for example 1 hour or overnight) to soar into the pollutants. The chemical mixture and pollutants are then displaced out of the line. Water is then displaced through the line to achieve rinsing, so that there is no residual chemical present. Beer is then reintroduced into the line. This is then repeated for the next line. Therefore a sequential process of filling, soaking and rinsing takes place. This is a “process” and requires no special product or equipment other than liquid chemical.

Various products and special equipment are also available. A patent search and web search revealed a number of products. Most use liquid chemicals which are fed into a chemical dosing pump device such as a centrifugal pump. The devices often include valves and timers or other control devices. The chemicals can include those listed in the previous point. One “non-chemical” product purports to use audio frequencies to achieve the cleaning. Compressed air projectile cleaners are also available.

Acidic and alkaline chemicals are the most common cleaning methods. They are consumables and therefore they need to be frequently purchased, transported and stored and dispensed. This creates significant on-going purchasing and logistics costs.

The chemicals are hazardous in nature. This creates occupational health and safety problems, during transport, storage and handling. Handling may include the need to pour between vessels and to dilute with water, often in a semi-confined space such as a basement, and often in an unsupervised situation.

The chemicals require a rinsing stage after the line is cleaned. This is often imprecise and the operator is not certain if sufficient rinsing has taken place. If rinsing is incomplete, the reintroduced beer suffers “off-tastes” or may be unhealthy.

The chemicals and pollutants, following cleaning, need to be removed from the line and disposed of. Typically they should not be run to the drain or sewer, because they are toxic. If they are not, then alternative disposal costs are high. If they are, then the operator is liable to be breaking the law.

The chemicals often require a lengthy soaking time. Therefore labour costs of the operator can be high as many organisations have many lines.

Some chemicals do not clean efficiently or purify efficiently, especially in the case of beer, milk and wine where yeast, beer-stone, milk-stone and wild yeasts may not be completely removed.

Some chemicals are excessively corrosive, and this is amplified where long soaking times are required.

It is an object of this invention to overcome one or more of the above problems associated with the cleaning of beverage lines.

A further object of the invention is to provide a system for cleaning and purifying beverage lines in which consumables are not required to be purchased and in which no polluting or hazardous materials are used which require correct and legal disposal after use.

### BRIEF STATEMENT OF THE INVENTION

Thus there is provided according to the invention a method of cleaning and purifying beverage lines and vessels including the steps of providing a flow of water through the beverage lines or into the vessel, electrically producing oxidants by passing air through an oxdising chamber such as a corona discharge chamber, mixing the oxidants with the flow of water whereby the oxidants in the water passing through the fines or vessel remove contaminants and/or kill microorganisms in the lines or vessels.

Also there is provided according to the invention a method of cleaning and purifying beverage lines and vessels, including the steps of providing a flow of water through the lines and vessel and producing ozone and/or hydroxyl radicals in the water flowing through the line to react with and remove contaminants in the lines or vessels.

Additionally there is provided according to the invention a method of cleaning and purifying beverage lines or vessels including the steps of providing a flow of water through the lines or vessels, passing air which contains oxygen and water vapour through an oxidising chamber to produce one or more oxidants in the form of ozone, hydrogen peroxide, hydroxyl radicals, hydroxyl ions, atomic oxygen, and atomic oxygen ions and injecting and mixing the oxidants in the flow of water through the line or vessel

There is also provided apparatus for cleaning and purifying beverage lines and vessels, said apparatus including an inlet connection to a supply of water, an outlet to be connected to a line to be cleaned or to be used to transfer to a vessel, an air inlet, an oxidant or ozone generator having an inlet connected to the air inlet, and an outlet connected to a passage between the water inlet and outlet whereby the products from the oxidant or ozone generator are passed into and mixed with the water to clean and purify the beverage lines or vessel.

### BRIEF DESCRIPTION OF THE DRAWINGS

In order to more fully describe the invention reference is now made to the accompanying drawings in which;

FIG. 1 is a general view of a typical beverage line system with the addition of the invention,

FIG. 2 is a view of one embodiment of the invention,

FIG. 3 is a further embodiment of the invention, and

FIG. 4 is a further embodiment of the invention.

### DESCRIPTION OF THE PREFERRED EMBODIMENT

A product is used to create strong oxidants from air. These oxidants are created by using electrical energy, such as by passing air which may contain water vapour through a corona discharge field. The oxidants which are created may include one or more of the following: ozone (triatomic oxygen), hydroxyl radical, hydroxyl ion, hydrogen peroxide, atomic oxygen, atomic oxygen ion, diatomic oxygen ion, hydrogen ions, nitrogen ions and similar. These oxidants are then dissolved into water by using a contact mechanism such as a venturi. This mixture of “oxidants in water” or “oxidised water” then exits the product and flows through the beverage line. In the beverage line itself, there may be a further phenomenon where ozone reacts with intermediary oxidants such as hydrogen peroxide and this creates further hydroxyl radicals.

The oxidised water may include:

i. Some oxidants which are properly dissolved in the water and are effectively in the liquid phase.

ii. Some oxidants which are not dissolved and are still in the gas phase, and for example may be seen as bubbles in the line. The beverage line is a closed system, and therefore oxidants which do not dissolve cannot escape from the system and are maintained in the line as bubbles until they vent from the tap at the end of the line. Alternatively the bubbles can be removed before exiting the product.

iii. Some residual air (diatomic oxygen molecules and nitrogen molecules and water vapour) which are present because the efficiencies of the air preparation devices are less than 100%.

The oxidised water both cleans and purifies pollutants from the inside of the line:

i. Cleaning is by a process of oxidation of inorganic and non-living organic substances on the line surfaces, also by a process of killing microorganisms which act as a substrate for other pollutants on the line, and also by a process of friction where the oxidised water flows past the line surfaces.

ii. Purification is by a process of the oxidants causing denaturing of the protein structure in micro-organisms and thereby killing them.

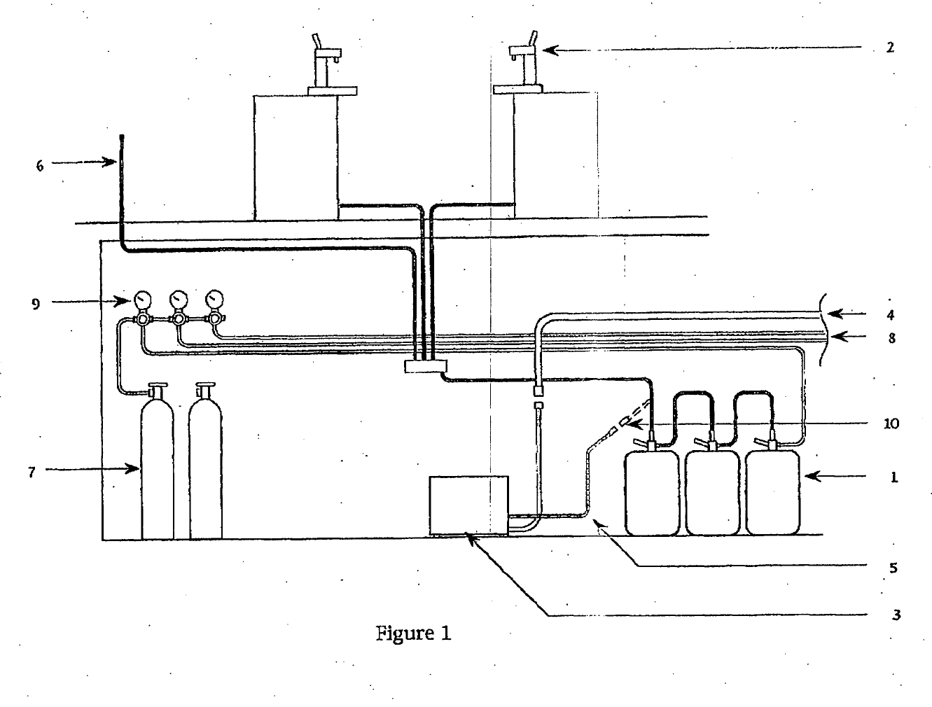

FIG. 1 shows a typical beverage line system comprising beer kegs 1 in a hotel basement with beer taps 2 at the floor above. The invention (the beverage purifier) 3 is placed at any convenient location, such as the basement. Its water inlet 4 is connected to mains water or a tank or vessel of water. The outlet 5 from unit 3 is connected to the beer lines 6 to be cleaned and purified. Various forms of connection devices 10 may be utilised, such as quick disconnect fittings or permanent connection manifolds with diversion valves. Electricity is also connected. No chemicals or consumables are required. Also shown are the gas cylinders 7, gas lines 8 and gas regulators 9. Therefore the installation is simple.

The water flow rate through the oxidation unit and the lines is low, for example 1 liter per minute per line. Multiple lines may be cleaned and purified at once, for example 8 lines at once which may result in 8 liters per minute of water being oxidised by the beverage purifier. The water is designed to flow continuously rather than the line being filled and soaked. The treatment time can vary, but one example is 1 hour per line, which results in only 60 liters of water being used per line per cleaning cycle. The waste water can be run from the beer tap directly to a bar sink below it, or to a small collection trough or bucket which in turn can have a line running from it to the sink. Multiple lines can be cleaned at once with the invention.

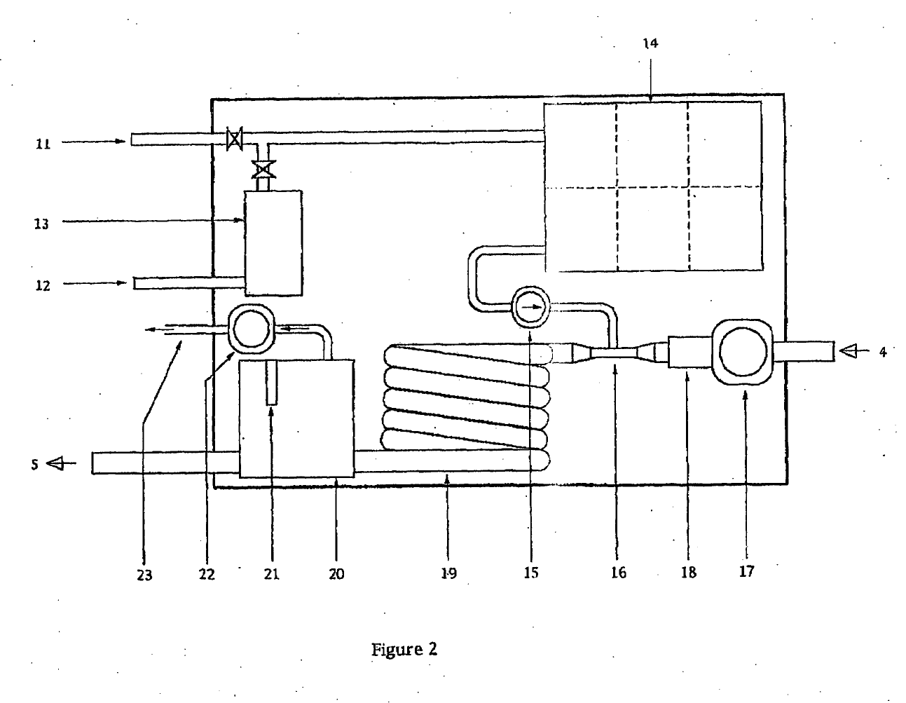

FIG. 2 shows a first embodiment of the invention. The air feedstock may be supplied from the ambient air through air inlet 11 and flow directly to an oxidising chamber 14. Ambient air contains some natural water vapour. Alternatively the air feedstock may be supplied from ambient air through alternative air inlet 12 and then pass through a dryer 13 so as to remove the water vapour before it passes to the oxidising chamber 14. The oxidising chamber utilises a corona discharge field to create strong oxidants. The oxidants are in the gas phase and may also be in the aqueous phase as a vapour or liquid aerosol. They then pass through a gas solenoid valve 15 which serves as a backflow prevention device and then to a contactor 16. The contactor is preferably a venturi injector which efficiently dissolves the oxidants into the main water flow.

The main water flow enters through water inlet 4 and then passes through water solenoid valve 17. The water feedstock may be from a pressurised mains system or it may be from a tank or keg or dam in which case a water pump may be included with the beverage purifier product. The solenoid 17 serves both as a backflow prevention device and also as an automatic method of activating the Beverage Purifier's electrics when connected to the flow switch 18, from which it receives an electrical signal. After the oxidants have been contacted with the water, they are mixed in the water by the mixing coil 19.

The oxidised water in the mixing coil contains some oxidants which are dissolved and some which are still in the form of bubbles. The oxidised water then passes through a degasser chamber 20 where the bubbles are separated from the water. The bubbles are expelled as gas to the vent outlet 23, whilst the oxidised water leaves the product at the oxidised water outlet 5. One form of the degasser chamber comprises a pressure vessel or tank into which the oxidised water enters, and thus the water velocity slows and allows bubbles to rise to the surface of the water, which creates a gaseous space at the top of the vessel. As this gas builds up, the water level in the vessel reduces and a float switch 21 sends an electric signal to a degasser solenoid 22 which opens and allows the gas to vent to outlet 23, until the float switch moves the solenoid back to the closed position. Thus the water which exits the product at water outlet 5 is highly oxidised but does not contain bubbles. Water outlet 5 is connected to the beverage line as previously described in reference to FIG. 1.

If the oxidised water contained bubbles then many practical problems can result. First, flow of the water through multiple beverage lines can be very uneven, resulting in inadequate cleaning and purifying of some of the lines. Second, beer line systems often include devices in the line to remove froth from beer, sometimes called defobbers. These devices also act to separate gas from liquid, and thus they soon fill up with gas and thus they are not cleaned or purified. Third, the lines can fill up with gas and cause air packets at some points, thus stopping water flow and even preventing beverage flow entirely when beverage is reconnected. Also, when the beverage liquid is reconnected, spluttering of the lines can occur, and frothing of the beverage can result as liquid and gas exit the taps.

The vent outlet 23 contains ozone gas. It may be connected to a tube which runs to a safe position away from operators. Or it may connect to an ozone destruction device such as adsorption media or a catalyst. Or preferably it is connected back into the gas flow, so that the ozone is used efficiently. It may be connected into the gas line just upstream of solenoid 15, or connected to a second gas port on contactor 16, or into the gas port of a second contactor which may be positioned upstream or downstream of the first contactor.

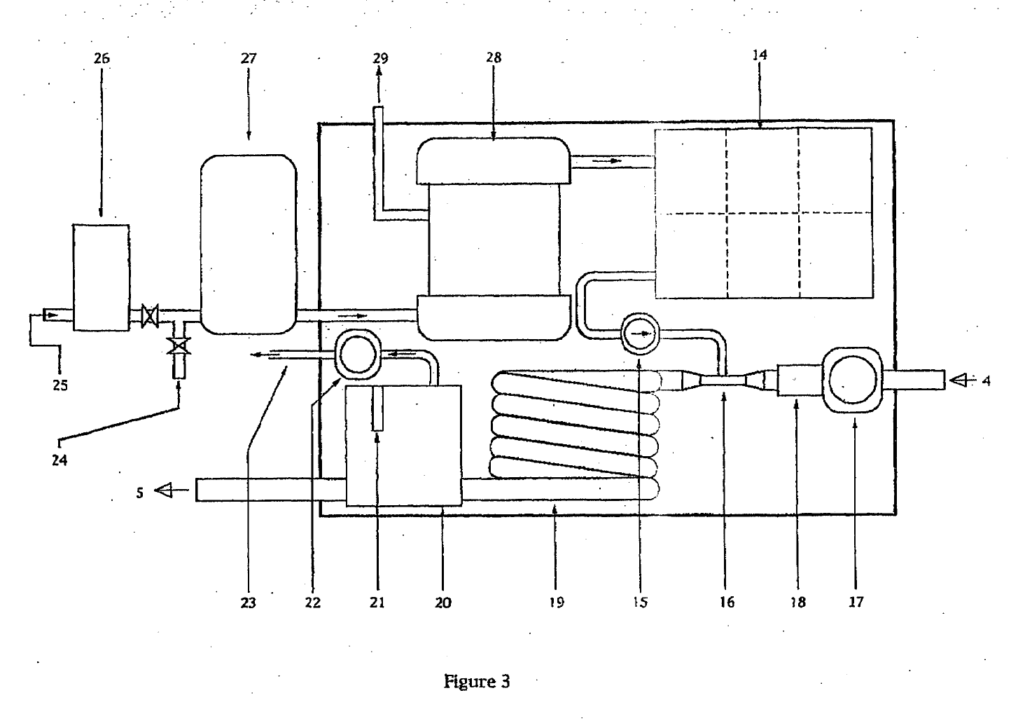

FIG. 3 shows a further form of the invention to increase the concentration of oxidants, by using an oxygen concentrator device, also known as an oxygenator. The air feedstock may be supplied from the ambient air through inlet 24 and then passes through an air preparation system, such as a compressor 27 through a tube to an oxygenator 28 to remove nitrogen 29 and achieve a high oxygen concentration. Alternatively the air feedstock may be supplied from ambient air through alternative air inlet 25 and then pass through a dryer 26 before it passes to the compressor 27. The output from the oxygenator passes to the oxidising chamber 14. The remainder of the beverage purifier system is then as was previously described in reference to FIG. 2.

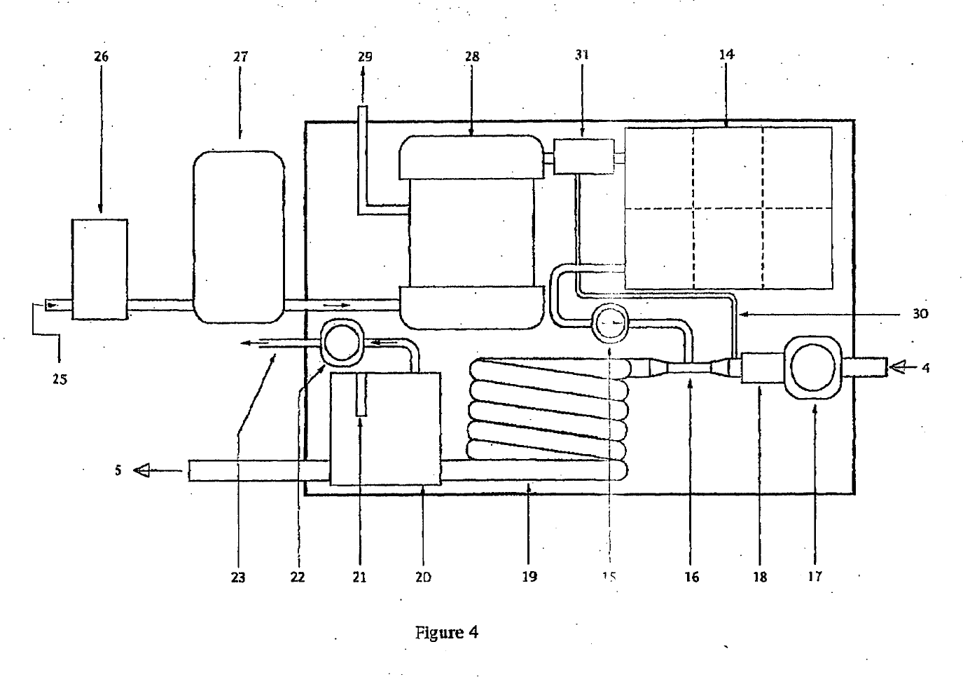

FIG. 4 shows a further form of the invention to create high concentrations of oxidants in the oxidant outlet, and also to optimise the efficiency and life of the product. The feedstock air enters the air inlet 25 and is dried 26 before being compressed 27 and oxygenated 28. The gas therefore mainly comprises dry oxygen. However before this gas flows into the oxidation chamber 14 it is humidified by a humidifier device 31. A flow of water is bled from the main water flow through fine 30 to the humidifier device 31 which mixes water in aerosol or droplet or vapour form into the gas which is flowing from the oxygenator to the oxidation chamber. The humidifier device preferably comprises a membrane contact device, which allows pressurised oxygen. Thus water vapour or aerosol (H2O) and oxygen (O2) and a minor quantity of residual air pass into the oxidation chamber. The remainder of the beverage purifier system is then as was previously described in reference to FIG. 2.

It has been proved by tests and investigation that the invention as described above can create two sets of oxidants depending upon the air inlets used.

i. In FIG. 2, when air inlet 12 is used, the feedstock air is dried, water vapour is removed and oxygen and nitrogen remain. Thus the resultant feedstock does not contain hydrogen atoms. The main oxidant then created by the oxidising chamber is ozone in medium concentrations.

ii. In FIG. 2, when air inlet 11 is used, the feedstock air contains water vapour, oxygen and nitrogen. The main oxidants created by the oxidising chamber are ozone in the gas phase in medium concentrations, and hydrogen peroxide in the aqueous phase and hydroxyl radicals, both in significant but relatively low concentrations.

iii. In FIG. 3, when air inlet 25 is used water vapour is removed by the dryer, nitrogen is removed by the oxygenator, and high concentrations of oxygen remain. The main oxidant then created by the oxidising chamber is ozone in high concentrations.

iv. In FIG. 3, when air inlet 24 is used, nitrogen is removed but water vapour and high concentrations of oxygen remain. The main oxidants created by the oxidising chamber are ozone in the gas phase in high concentrations, and hydrogen peroxide in the aqueous phase and hydroxyl radicals, both in medium concentrations.

v. In FIG. 4, air inlet 25 is used. The dryer removes water vapour, the oxygenator removes nitrogen and high concentrations of oxygen remain downstream of the oxygenator 28. The humidifier then adds water vapour in a fine aerosol form. The main oxidants created by the oxidising chamber are ozone in the gas phase in high concentrations, and hydrogen peroxide in the aqueous phase and hydroxyls, both in high concentrations.

The advantage of creating hydroxyls is that they are very strong oxidants and provide an advanced oxidation process. For example, measured in millivolts, the oxidation potential of chlorine gas is 1.36, ozone is 2.07 and the hydroxyl radical is 2.80. There are many substances, including synthetic organic chemicals, which have a slow reaction rate with ozone but a fast reaction rate with hydroxyls, and in such instances hydroxyls are superior oxidants. Hydroxyls have a short half life, being a fraction of a second whilst ozone has a longer half life being up to 30 minutes in clean water. Therefore for microorganism disinfection, where a residual oxidant level is required for a period of time, ozone is a superior oxidant. Other examples also exist where either hydroxyls or ozone or both, can be chosen to provide the optimum oxidant regime.

Further, the invention is able to create hydroxyls in a downstream pipe, connected to the oxidised water outlet 5. In FIG. 4 for example, wet oxygen is used as feedstock to the oxidising chamber which creates ozone in the gas phase and hydrogen peroxide in the aqueous phase, and also creates some hydroxyl radicals. The ozone and the hydrogen peroxide are created independently from each other and at the same time and in a single operation, whilst the feedstock is passing through the discharge gap in the emitter. The ozone and hydrogen peroxide are then mixed into the main water flow at the contactor. The hydrogen peroxide then acts as an intermediary. It gradually reacts with some of the ozone, in this downstream and hydroxyls which are created or generated in the downstream line, such as in the beverage line. If the hydroxyls were only created in the oxidising chamber itself, then they would not be able to do useful work in a downstream line, as they would disappear quickly due to their fast half life which is a fraction of a second. But because the invention allows them to be generated in a downstream line, this limitation is solved.

Ozone decomposes in water with a natural half-life. When it does so, hydroxyl radicals are generated as a transient by-product. However the process described above, involving hydrogen peroxide, is a separate phenomenon and involves the generation of larger quantites of hydroxyl radicals from a reaction between ozone and hydrogen peroxide.

The presence of water vapour in the discharge space of the oxidising chamber acts to reduce the ozone output rate and the ozone concentration which would otherwise be achieved if the space was dry. However this effect is counteracted by the formation of hydrogen peroxide which in turn enables the generation of larger quantities of hydroxyl radicals.

The oxidising chamber is designed so that it can create ozone and hydrogen peroxide and hydroxy, by receiving wet (humid) air or wet (humid) oxygen.

A corona discharge field is developed. Mains electrical input is transformed into the optimum combination of voltage, frequency and wave shape, so as to disassociate the diatomic oxygen and water vapour molecules into atomic oxygen and hydrogen, to then enable recombination into the required oxidants.

The invention is designed to minimise corrosion rates and to extend component life, for applications where hydroxyls and ozone are required and therefore wet air or wet oxygen feedstock is used.

i. In FIG. 2, when inlet 11 is used, water vapour and nitrogen flow through the corona field in the oxidising chamber. Trace levels of substances may form, such as nitric acid, which may gradually corrode the surfaces of components in the oxidising chamber which are in the gas stream, including stainless steels. One solution is to design the beverage purifier so that it is used for an intermittent duty only, so that resultant product life is satisfactory, Alternatively, the oxidising chamber is designed so that it is non-corrosive. The oxidising chamber comprises emitters, power sources, printed circuit boards, etc. There may be multiple emitters, in parallel or in series, so as to achieve the desired oxidant output and concentrations. A corona field is created in the emitter and the feedstock flows through this field. The emitters include a high voltage electrode, an earthed electrode and a dielectric. The electrodes may be made of metals including stainless steels or other materials which are electrically conductive and such materials are corrosive to some extent. The dielectric is made of silicon based materials, including glass, which have high corrosion resistance. The emitter design is laminated so that the a dielectric lies on top of the high voltage electrode, or the high voltage electrode is encapsulated in a dielectric. Therefore this electrode is not adjacent to the feedstock flow and thus it does not corrode. In addition, or alternatively, the earthed electrode can also be laminated by positioning a second dielectric against it, or it can be encapsulated by the dielectric. Thus one or both electrodes can be completely removed from the feedstock flowing through the emitter and thus corrosion is reduced and the efficiency of the oxidising chamber is maintained.

ii. In FIG. 3 there is an oxygenator which removes nitrogen and thus substances such as nitric acid do not form in the oxidising chamber and thus corrosion is controlled. However the water vapour flows through the oxygenator which can damage the molecular sieve media in it and reduce media life or reduce the efficiency of oxygen concentration. One solution is to design the beverage purifier so that it is used for an intermittent duty only, so that resultant life and efficiency are satisfactory. Alternatively, the oxygenator is designed so that it includes an excessive amount of molecular sieve media, and where this media may be easily replaced at a regular service interval.

iii. FIG. 4 shows a preferred configuration of the beverage purifier. The dryer 26 removes water vapour so that the molecular sieve material in the oxygenator 28 is not damaged and so that oxygen concentration efficiency is maintained. The oxygenator removes, nitrogen so that substances such as nitric acid do not form in the oxidising chamber. The water vapour is added to the system at the optimum location, namely the humidifier 31, so that hydroxyls can be created either in the oxidising chamber itself or in downstream lines via the hydrogen peroxide intermediary The oxidising chamber can also utilise an emitter design with laminated electrodes as previously described, so as to provide an extra level of corrosion protection.

The Invention may be configured by using various component options and configurations, including:

The dryer components 13 and 26 may comprise desiccant media, with or without a regenerative heater circuit, or may be a refrigerative dryer, or may be a coelescer or water trap device or mist filter. A particulate fiber may be added to remove pollutants to protect the compressor and oxygenator and oxidising chamber. The oxygenator may utilise a molecular sieve, or pressure swing absorption design, or membrane design. The compressor 27 and oxygenator 28 may be replaced with bottled oxygen. The humidifier may utilise a porous membrane or any other method which allows the oxygen to become saturated with water at up to 100% relative humidity.

The oxidising chamber may comprise corona discharge, plasma discharge, silent electrical discharge, dielectric barrier AC discharge or ultra-violet radiation or other electrical methods of creating oxidants. The emitters in the oxidising chamber may comprises electrodes which are tubular in shape or which utilise a parallel plate shape The electrodes may be solid material or may be granular. The contactor may comprise a venturi or a porous diffuser which bubbles into a contact tower or pipe, or a membrane device.

Or the contactor may utilise a peristaltic pump through which the oxidised gas passes so that this pump forces the gas through a porous diffuser into the water flow. Or if mains water pressure is not used, then a dual head peristaltic pump may be utilised, where one pump head creates pressurised water for the purpose of the main water flow and the other head creates pressurised oxidised gas which is then forced through a porous diffuser into the water flow. The mixing coil may be replaced by or used in conjunction with a static mixing device placed in a section of pipe. The product can be configured with or without the alternative air inlets previously described, and preferably would only incorporate the inlets which result in hydroxyls and ozone being created rather than ozone alone. The oxidising chamber includes multiple emitters and these emitters are preferably each encapsulated in a potting compound such as epoxy. This provides a method of achieving low electrical magnetic interference, safe electrical insulation and waterproofing.

The advantages of the invention include the following:

i. Hydroxyl radicals and ozone are created in the downstream lines which are connected to the beverage purifier. Thus these oxidants can do useful work in the line belt such as cleaning and purifying the surface of the line, even in the case of a long line which may be several hundred meters long. The hydroxyl radicals are created in the line itself, due to a reaction between the ozone and intermediary oxidants such as hydrogen peroxide, which are previously created in the oxidising chamber of the product and then mixed into the main water flow. The hydroxyl radicals are very strong oxidants which are ideal for oxidising inorganics and non-living organic whilst the ozone creates a temporary residual oxidation level which is ideal for killing microorganisms.

ii. The process is an all-electric advanced oxidation process. There are no chemicals or consumables. This creates significant on-going purchasing and logistics savings. The combination of this all-electric process together with hydroxyls being generated downstream in a line (as per point I above), is a unique and innovative combination.

iii. There are no hazardous chemicals. This creates occupational health and safety advantages, during transport, storage and handling.

iv. The need to rinse the line after treatment is reduced and in some cases eliminated. This is because the oxidants are made from air. “Left over” oxidants mainly revert back to oxygen and water vapour, leaving no chemical residue.

v. The chemicals and pollutants, following cleaning, can usually be run to waste. They do not contain acids or alkalis. Typically they can be run to the drain or sewer, because this is safe and legal.

vi. No soaking time is required. Rather, the oxidised water is run continuously through the line at a slow flow rate. Therefore labour costs of the operator can be reduced. The product can be set to run on a timer and then left to undertake automatic treatment. There are no intermittent stages and therefore an operator does not need to be present during the treatment

vii. The oxidants clean efficiently and purify efficiently, especially in the case of beer, milk, wine, juice and water where yeasts, beer-stone, milk-stone, wild yeasts and other surface pollutants may be present. A wide range of micro-organisms, including bacteria, are killed.

viii. The oxidants do not excessively corrode the lines or beverage fittings.

ix. Instruments can be used to give a sufficiently precise indication of whether the oxidation process is taking place. One method is to use a redox or ORP meter, also known as an oxidation reduction potential meter.

Larger versions of the inventions can be used for multiple beverage line applications or for large diameter pipes. The invention can also be used for cleaning vessels and tanks rather than lines. Examples include wine barrels and vats, in which case the beverage line purifier is connected to a short line which then runs into the barrel or vat, or the line connects to a water spray device which is located in the barrel or vat.

The invention can be applied to any beverage line or vessel, including beer, soft-drink, post-mix syrup, milk in diaries, wine in wineries, orange juice and water. Or it can be applied to non-beverage food processing lines and vessels, food manufacturing lines and vessels, etc, which can be treated with oxidised water to attain cleaning and purification.

Thus it can be seen that beverage and food lines can be effectively cleaned and purified without the use of chemicals or projectile cleaning devices. By connecting a unit which provides an advanced oxidation process and passing the oxidised water through the lines an effective and safe system of cleaning and purification is provided.

Although alternate forms of the invention have been described in some detail it is to be realised the invention is not to be limited thereto but can include variations and modifications failing within the spirit and scope of the invention.

1. A method of cleaning and purifying beverage lines and vessels, including the steps of providing a flow of water through the lines and vessel and producing oxidants in the water flowing through the line to react with and remove contaminants in the lines or vessels, where these oxidants are generated from molecules of air and/or water and thus contain oxygen and/or hydrogen atoms only, and include oxidants other than ozone, such as hydroxyl radicals or hydrogen peroxide.

2. A method of cleaning and purifying beverage lines or vessels including the steps of providing a flow of water through the lines or vessels, passing air which contains oxygen and water vapour through an oxidising chamber to produce oxidants in the form of hydrogen peroxide and one or more of hydroxyl radials, ozone, hydroxyl ions, atomic oxygen and atomic oxygen ions and injecting and mixing the oxidants in the flow of water through the line or vessel.

3. A method of cleaning and purifying beverage lines or vessels as defined in claim 2 wherein ozone and hydrogen peroxide are produced in an oxidising chamber and then injected into water wherein hydrogen peroxide then acts as an intermediary and reacts with the ozone to form hydroxyl radicals in the line downstream of the point of injection into the flow of water, including in the beverage lines or vessels through which the oxidised water flows.

4. A method of cleaning and purifying beverage lines and vessels as defined in any one of claims 1, 2, or 3 including the step of generating the oxidants by an electrical means only.

5. A method of cleaning and purifying beverage lines or vessels as defined in any one of claims 1, 2 or 3, including the steps of passing air through an ozone generator, then injecting and mixing the ozone into water flowing through beverage lines or vessels to clean and purify the beverage lines or vessels.

6. A method of cleaning and purifying beverage lines or vessels as defined in any one of claims 1, 2 or 3, including the steps of passing a flow of water through the beverage line or into the vessel, drying and compressing air, passing the dried compressed air through an oxygenator to remove nitrogen from the air, adding water in the form of aerosol, vapour or mist or droplets into the gas, passing this gas which has high concentrations of oxygen and water vapor through an electrical oxidising chamber and injecting the resultant oxidants into the flow of water through the lines.

7. Apparatus for cleaning and purifying beverage lines and vessels, said apparatus including an inlet connection to a supply of water, an outlet to be connected to a line to be cleaned or to be used to transfer to a vessel, an air inlet, an oxidant or ozone generator having an inlet connected to the air inlet, and an outlet connected to a passage between the water inlet and outlet whereby the products from the oxidant or ozone generator are passed into and mixed with the water to clean and purify the beverage lines or vessel.

8. Apparatus as defined in claim 7 characterised by an oxygenator positioned in the air line prior to the oxidant or ozone generator whereby oxygen enriched air is passed to the oxidant or ozone generator to produce ozone and/or hydroxyl radicals generated down stream in the beverage line.

9. An apparatus as defined in claim 7 or claim 8 characterised by an air drier positioned in the air inlet line.

10. An apparatus as defined in claim 9 characterised by an air compressor to pressurize the inlet air.

11. An apparatus as defined in claim 8 characterised by a humidifer positioned in the gas line between the oxygenator and the oxidant or ozone generator to humidify the gas by water spray, water aerosol, mist, droplet or stream.

12. An apparatus as defined in claim 7 characterised in that the oxidised water flow passes through a mixer prior to entering the beverage lines.

13. An apparatus as defined in claim 7 characterised by passing the oxidised water flow through a degasser to remove undissolved gasses and bubbles prior to entering the beverage lines.

14. An apparatus as defined in claim 7 characterised in that the beverage lines are selected from lines carrying beer, wine, milk, juice, water, non-alcoholic beverages and syrups.
